(19) United States
(12) Patent Application Publication
(10) Pub. No.: US 2025/0311984 A1
(43) Pub. Date: Oct. 9, 2025

(54) METHOD AND SYSTEM FOR REMOTE PHSICAL EXAMINATION

(71) Applicant: SHANGHAI UNITED IMAGING HEALTHCARE CO., LTD., Shanghai (CN)

(72) Inventor: Chunjiang YAO, Shanghai (CN)

(73) Assignee: SHANGHAI UNITED IMAGING HEALTHCARE CO., LTD., Shanghai (CN)

(21) Appl. No.: 19/243,619

(22) Filed: Jun. 19, 2025

Related U.S. Application Data
(63) Continuation of application No. PCT/CN2024/088354, filed on Apr. 17, 2024.

(30) Foreign Application Priority Data
Apr. 17, 2023 (CN) 202310411909.5

(51) Int. Cl. A61B 5/00; G16H 10/60; G16H 15/00; G16H 40/67; G16H 50/20; G16H 80/00

(52) U.S. Cl. CPC A61B 5/7465; G16H 10/60; G16H 15/00; G16H 40/67; G16H 50/20; G16H 80/00

(57) ABSTRACT

A system for remote physical examination may include an operator end and a patient end. The operator end may include a first extended reality device. The patient end may include a physical examination device. The physical examination device may be configured to obtain physical information of a patient, send the physical information to the first extended reality device, receive examination information from the extended reality device, and perform a second target examination operation on the patient based on the examination information. The first extended reality device may be configured to generate a digital twin model of the patient based on the physical information; cause the digital twin model to be displayed, determine the examination information based on a first target examination operation performed, based on the digital twin model through the first extended reality device, by an operator, and send the examination information to the physical examination device.

### CROSS-REFERENCE TO RELATED APPLICATIONS

This application is a continuation of International Application No. PCT/CN2024/088354, filed on Apr. 17, 2024, which claims priority to Chinese Patent Application No. 202310411909.5 filed on Apr. 17, 2023, the contents of which are hereby incorporated by reference.

### TECHNICAL FIELD

The present disclosure generally relates to the medical field, and in particular, to systems and methods for remote physical examination.

### BACKGROUND

In real life, due to various reasons, the patient and the doctor may be separated from each other, and the doctor cannot perform a physical examination on the patient face to face. Therefore, it is desired to provide systems and methods for remote physical examination.

### SUMMARY

According to an aspect of the present disclosure, a method for remote physical examination implemented on a physical examination device provided at a patient end may include one or more of the following operations. One or more processors may obtain physical information of a patient. The one or more processors may send the physical information to a first extended reality device provided at an operator end, so that the first extended reality device generates a digital twin model of the patient based on the physical information. The one or more processors may receive examination information from the first extended reality device. The examination information may relate to a first target examination operation performed, based on the digital twin model through the first extended reality device, by an operator. The one or more processors may perform a second target examination operation on the patient based on the examination information.

In some embodiments, the physical examination device may include a sensor configured to acquire the physical information.

In some embodiments, the physical examination device may include a first wearable sensory device that is worn by the patient and configured to perform the physical examination on the patient.

In some embodiments, the physical examination device may include a mechanical arm configured to perform the physical examination on the patient.

In some embodiments, the examination information may include at least one examination position of the first target examination operation and at least one of a type, a direction, or a first intensity of the first target examination operation.

In some embodiments, the examination information may include at least one examination position of the first target examination operation and a first intensity of the first target examination operation. The operation of performing the second target examination operation on the patient based on the examination information may include: determining whether the first intensity is within a protection range; in response to determining that the first intensity is outside the protection range, determining a second intensity based on the protection range; and performing the second target examination operation on the patient based on the second intensity.

In some embodiments, the protection range may be determined using a protection range determination model based on at least one of patient information or medical records of the patient.

In some embodiments, the examination information may include at least one examination position of the first target examination operation and at least one of a type of the first target examination operation, a direction of the first target examination operation, or a second intensity corresponding to the at least one examination position. The second intensity may be determined, by the first extended reality device, based on a first intensity of the first target examination operation and a protection range.

In some embodiments, the digital twin model may include visualization of at least one diseased part of the patient, the at least one diseased part including at least one of a suspected diseased part or a definite diseased part.

In some embodiments, the suspected diseased part may be predicted using a diseased part prediction model based on at least one of patient information or medical records of the patient.

According to another aspect of the present disclosure, a device for remote physical examination provided at a patient end may include one or more storage devices and one or more processors configured to communicate with the one or more storage devices. The one or more storage devices may include a set of instructions. When the one or more processors executing the set of instructions, the one or more processors may be directed to perform one or more of the following operations. The one or more processors may obtain physical information of a patient. The one or more processors may send the physical information to a first extended reality device provided at an operator end, so that the first extended reality device generates a digital twin model of the patient based on the physical information. The one or more processors may receive examination information from the first extended reality device. The examination information may relate to a first target examination operation performed, based on the digital twin model through the first extended reality device, by an operator. The one or more processors may perform a second target examination operation on the patient based on the examination information.

According to yet another aspect of the present disclosure, a device for remote physical examination provided at a patient end may include: a first obtaining module configured to obtain physical information of a patient; a first transmission module configured to send the physical information to a first extended reality device provided at an operator end, so that the first extended reality device generates a digital twin model of the patient based on the physical information; a first receiving module configured to receive examination information from the first extended reality device; and an examination module configured to perform a second target examination operation on the patient based on the examination information. The examination information may relate to a first target examination operation performed, based on the digital twin model through the first extended reality device, by an operator.

According to yet another aspect of the present disclosure, a non-transitory computer readable medium may comprise at least one set of instructions. The at least one set of instructions may be executed by one or more processors of a device provided at a patient end. The one or more processors may obtain physical information of a patient. The one or more processors may send the physical information to a first extended reality device provided at an operator end, so that the first extended reality device generates a digital twin model of the patient based on the physical information. The one or more processors may receive examination information from the first extended reality device. The examination information may relate to a first target examination operation performed, based on the digital twin model through the first extended reality device, by an operator. The one or more processors may perform a second target examination operation on the patient based on the examination information.

According to yet another aspect of the present disclosure, a method for remote physical examination implemented on a first extended reality device provided at an operator end may include one or more of the following operations. One or more processors may obtain physical information of a patient. The one or more processors may generate a digital twin model of the patient based on the physical information. The one or more processors may cause the digital twin model to be displayed. The one or more processors may determine examination information based on a first target examination operation performed, based on the digital twin model, by an operator. The one or more processors may send the examination information to a physical examination device provided at a patient end, so that the physical examination device performs a second target examination operation on the patient based on the examination information.

In some embodiments, the examination information may include at least one examination position of the first target examination operation and at least one of a type, a direction, or a first intensity of the first target examination operation.

In some embodiments, the examination information may include at least one examination position of the first target examination operation and at least one of a type of the first target examination operation, a direction of the first target examination operation, or a second intensity corresponding to the at least one examination position. The operation of determining the examination information based on the first target examination operation may include: determining whether a first intensity of the first target examination operation is within a protection range; and in response to determining that the first intensity is outside the protection range, determining the second intensity based on the protection range.

In some embodiments, the protection range may be determined using a protection range determination model based on at least one of patient information or medical records of the patient.

In some embodiments, the first extended reality device may include a virtual touch emulating device worn by at least one hand of the operator. The one or more processors may simulate, based on the examination information, a tactile sensation obtained by the operator when the first target examination operation or the second target examination operation may be performed on the patient.

In some embodiments, the operator may perform the first target examination operation on the digital twin model.

In some embodiments, the first extended reality device may include a second wearable sensory device.

In some embodiments, the operator may perform the first target examination operation on the second wearable sensory device.

In some embodiments, the digital twin model may include visualization of at least one diseased part of the patient, the at least one diseased part including at least one of a suspected diseased part or a definite diseased part.

In some embodiments, the suspected diseased part may be determined using a diseased part prediction model based on at least one of patient information or medical records of the patient.

According to another aspect of the present disclosure, a device for remote physical examination provided at an operator end may include one or more storage devices and one or more processors configured to communicate with the one or more storage devices. The one or more storage devices may include a set of instructions. When the one or more processors executing the set of instructions, the one or more processors may be directed to perform one or more of the following operations. The one or more processors may obtain physical information of a patient. The one or more processors may generate a digital twin model of the patient based on the physical information. The one or more processors may cause the digital twin model to be displayed. The one or more processors may determine examination information based on a first target examination operation performed, based on the digital twin model, by an operator. The one or more processors may send the examination information to a physical examination device provided at a patient end, so that the physical examination device performs a second target examination operation on the patient based on the examination information.

According to yet another aspect of the present disclosure, a device for remote physical examination provided at an operator end may include: a second obtaining module configured to obtain physical information of a patient; a modeling module configured to generate a digital twin model of the patient based on the physical information; a display module configured to cause the digital twin model to be displayed; a third obtaining module configured to determine examination information based on a first target examination operation performed, based on the digital twin model, by an operator; and a second transmission module configured to send the examination information to a physical examination device provided at a patient end, so that the physical examination device performs a second target examination operation on the patient based on the examination information.

According to yet another aspect of the present disclosure, a non-transitory computer readable medium may comprise at least one set of instructions. The at least one set of instructions may be executed by one or more processors of a device provided at an operator end. The one or more processors may obtain physical information of a patient. The one or more processors may generate a digital twin model of the patient based on the physical information. The one or more processors may cause the digital twin model to be displayed. The one or more processors may determine examination information based on a first target examination operation performed, based on the digital twin model, by an operator. The one or more processors may send the examination information to a physical examination device provided at a patient end, so that the physical examination device performs a second target examination operation on the patient based on the examination information.

According to yet another aspect of the present disclosure, a system for remote physical examination may include an operator end and a patient end. The operator end may include a first extended reality device. The patient end may include a physical examination device. The physical examination device may be configured to obtain physical information of a patient, send the physical information to the first extended reality device, receive examination information from the extended reality device, and perform a second target examination operation on the patient based on the examination information. The first extended reality device may be configured to receive the physical information from the physical examination device, generate a digital twin model of the patient based on the physical information; cause the digital twin model to be displayed, determine the examination information based on a first target examination operation performed, based on the digital twin model through the first extended reality device, by an operator, and send the examination information to the physical examination device.

Additional features will be set forth in part in the description which follows, and in part will become apparent to those skilled in the art upon examination of the following and the accompanying drawings or may be learned by production or operation of the examples. The features of the present disclosure may be realized and attained by practice or use of various aspects of the methodologies, instrumentalities, and combinations set forth in the detailed examples discussed below.

### BRIEF DESCRIPTION OF THE DRAWINGS

The present disclosure is further described in terms of exemplary embodiments. These exemplary embodiments are described in detail with reference to the drawings. These embodiments are non-limiting exemplary embodiments, in which like reference numerals represent similar structures throughout the several views of the drawings, and wherein:

FIG. 1 is an exemplary block diagram of a system for remote physical examination according to some embodiments of the present disclosure;

FIG. 2 is a schematic diagram illustrating a front view and a cross-sectional view of an exemplary wearable sensory overall according to some embodiments of the present disclosure;

FIGS. 3A and 3B are schematic diagrams illustrating two exemplary mechanical arms according to some embodiments of the present disclosure;

FIG. 4 is a schematic block diagram illustrating an exemplary system for remote physical examination implemented on a patient end according to some embodiments of the present disclosure;

FIG. 5 is a schematic block diagram illustrating an exemplary system for remote physical examination implemented on an operator end according to some embodiments of the present disclosure;

FIG. 6 is a flowchart illustrating an exemplary process for remote physical examination implemented on a patient end according to some embodiments of the present disclosure; and

FIG. 7 is a flowchart illustrating an exemplary process for remote physical examination implemented on an operator end according to some embodiments of the present disclosure.

### DETAILED DESCRIPTION

In the following detailed description, numerous specific details are set forth by way of examples in order to provide a thorough understanding of the relevant disclosure. However, it should be apparent to those skilled in the art that the present disclosure may be practiced without such details. In other instances, well-known methods, procedures, systems, components, and/or circuitry have been described at a relatively high-level, without detail, in order to avoid unnecessarily obscuring aspects of the present disclosure. Various modifications to the disclosed embodiments will be readily apparent to those skilled in the art, and the general principles defined herein may be applied to other embodiments and applications without departing from the spirit and scope of the present disclosure. Thus, the present disclosure is not limited to the embodiments shown, but to be accorded the widest scope consistent with the claims.

The terminology used herein is for the purpose of describing particular example embodiments only and is not intended to be limiting. As used herein, the singular forms “a,” “an,” and “the” may be intended to include the plural forms as well, unless the context clearly indicates otherwise. It will be further understood that the terms “comprise,” “comprises,” and/or “comprising,” “include,” “includes,” and/or “including,” when used in this specification, specify the presence of stated features, integers, steps, operations, elements, and/or components, but do not preclude the presence or addition of one or more other features, integers, steps, operations, elements, components, and/or groups thereof.

It will be understood that the term “system,” “unit,” “module,” and/or “block” used herein are one method to distinguish different components, elements, parts, section or assembly of different level in ascending order. However, the terms may be displaced by another expression if they achieve the same purpose.

Generally, the word “module,” “unit,” or “block,” as used herein, refers to logic embodied in hardware or firmware, or to a collection of software instructions. A module, a unit, or a block described herein may be implemented as software and/or hardware and may be stored in any type of non-transitory computer-readable medium or another storage device. In some embodiments, a software module/unit/block may be compiled and linked into an executable program. It will be appreciated that software modules can be callable from other modules/units/blocks or from themselves, and/or may be invoked in response to detected events or interrupts. Software modules/units/blocks configured for execution on computing devices may be provided on a computer readable medium, such as a compact disc, a digital video disc, a flash drive, a magnetic disc, or any other tangible medium, or as a digital download (and can be originally stored in a compressed or installable format that needs installation, decompression, or decryption prior to execution). Such software code may be stored, partially or fully, on a storage device of the executing computing device, for execution by the computing device. Software instructions may be embedded in firmware, such as an EPROM. It will be further appreciated that hardware modules/units/blocks may be included of connected logic components, such as gates and flip-flops, and/or can be included of programmable units, such as programmable gate arrays or processors. The modules/units/blocks or computing device functionality described herein may be implemented as software modules/units/blocks, but may be represented in hardware or firmware. In general, the modules/units/blocks described herein refer to logical modules/units/blocks that may be combined with other modules/units/blocks or divided into sub-modules/sub-units/sub-blocks despite their physical organization or storage.

It will be understood that when a unit, engine, module or block is referred to as being “on,” “connected to,” or “coupled to,” another unit, engine, module, or block, it may be directly on, connected or coupled to, or communicate with the other unit, engine, module, or block, or an intervening unit, engine, module, or block may be present, unless the context clearly indicates otherwise. As used herein, the term “and/or” includes any and all combinations of one or more of the associated listed items.

These and other features, and characteristics of the present disclosure, as well as the methods of operation and functions of the related elements of structure and the combination of parts and economies of manufacture, may become more apparent upon consideration of the following description with reference to the accompanying drawings, all of which form a part of this disclosure. It is to be expressly understood, however, that the drawings are for the purpose of illustration and description only and are not intended to limit the scope of the present disclosure. It is understood that the drawings are not to scale.

The present disclosure provides a method and a system for remote physical examination. The method and the system rely on extended reality technology to display a digital twin model of a patient to an operator. In an operator end, the operator can comprehensively and intuitively understand the patient's physical condition through the digital twin model and perform a first target physical examination operation. Examination information related to the first target physical examination operation is transmitted to a patient end, so that a second target physical examination operation can be performed to the patient based on the examination information. It can be understood that the physical examination refers to an examination that achieves a purpose of medical examination by touching a surface of a human body. For example, the physical examination may include an operation of pressing the calf of a patient. The patient's feedback on the pressing operation (e.g., pain or not) may be used as a reference or basis for a doctor to make diagnostic opinions. In addition, the person who performs the physical examination does not necessarily have to be a doctor, but can also be other operators, e.g., other medical practitioners such as, technicians, nurses, physical therapists, etc. The person receiving the physical examination does not necessarily have to be a patient, but can also be other objects that are examined, such as people undergoing a test, simulated human bodies, etc. For the convenience of description, patients and doctors are taken as examples in the present disclosure.

The method and the system for remote physical examination provided in the present disclosure may be applied to various scenarios. For example, when there are insufficient local doctors in an area, patients in the area may receive remote physical examination from doctor in other area. As another example, if a patient has limited mobility or does not want to go out, the patient may receive remote physical examination from a doctor. As still another example, a patient may receive remote physical examination from a doctor when traffic between the patient and the doctor is controlled.

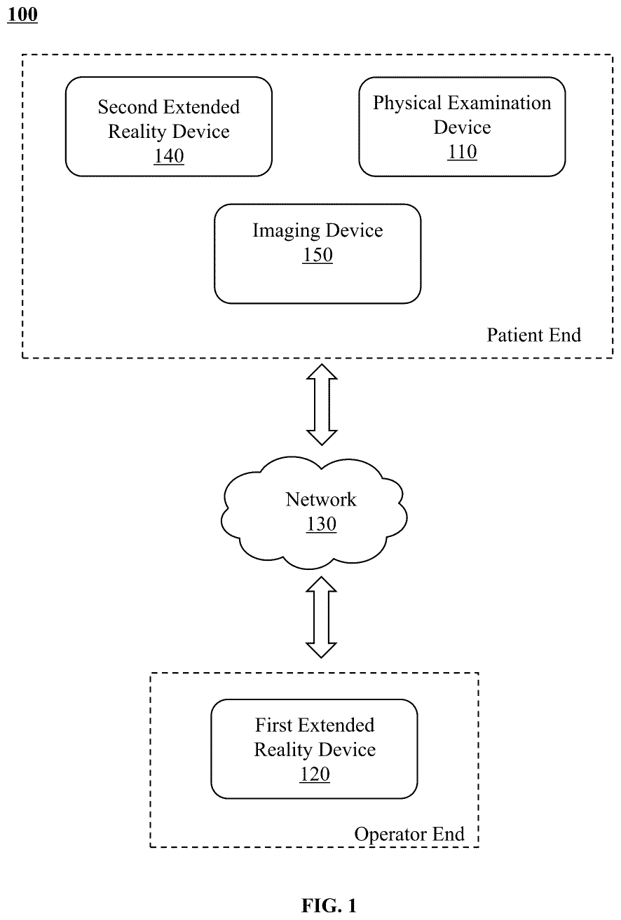

FIG. 1 is an exemplary block diagram of a system for remote physical examinations according to some embodiments of the present disclosure. As shown in FIG. 1, the system 100 may include a physical examination device 110, a first extended reality device 120, and a network 130. The physical examination device 110 may include a first controller and a first external device of the first controller. The first external device may include at least one sensor, a first wearable sensory device, at least one mechanical arm, or the like, or any combination thereof. The first extended reality device 120 may include a second controller and a second external device of the second controller. The second external device may include a head-mounted display device, a touch emulating device, a second wearable sensory device, or the like, or any combination thereof.

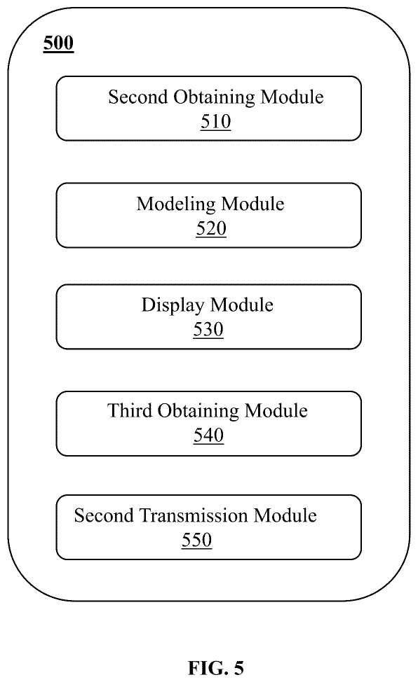

The physical examination device 110 may belong to a patient end. At least a part of the patient end (e.g. the first external device) may be regarded as a local device that is located at a place where the patient receives remote physical examination. For example, the first external device may locate at the patient's residence or a clinic in the patient's neighborhood. The first extended reality device 120 may belong to an operator end. At least a part of the operator end (e.g. the second external device) may be regarded as a local device that is located at a place where the operator performs remote physical examination. For example, the operator end may be located at a hospital, the operator's residence, or a clinic in the operator's neighborhood. In some embodiments, at least a part of the patient end and/or the operator end may be implemented on a remote server (e.g. a public cloud, a private cloud, or a hybrid cloud). For example, a modeling module 520 of the first extended reality device 120 as shown in FIG. 5 may be implemented on a remote server, and a local head-mounted display device of the first extended reality device 120 may receive a digital twin model generated by the reconstruction module from the remote server.

The physical examination device 110 may collect the patient's physical information and send the physical information to the first extended reality device 120. The patient's physical information may be used to generate a digital twin model of the patient. The digital twin model (digital twin for short) is a computer-generated (e.g. by the first extended reality device 120) virtual projection of a real-life human body.

The physical information may include one or more physiological indicators. In some embodiments, the physical information may include body sizes, posture, body temperature, heart rate, pulse, blood pressure, blood oxygen saturation, complexion, skin smoothness, smell, or the like, or any combination thereof. In some embodiments, the body sizes may include the height, the wingspan, the length of legs, the width of shoulders, the circumference of head, chest, waist, or hip, or the like, or any combination thereof.

In some embodiments, the physical examination device 110 may include at least one sensor configured to acquire the physical information. For example, as for body sizes/posture, coordinates of a plurality of pre-set reference points (e.g. joints) of the patient may be acquired by a position sensor, wherein the body sizes/posture of the patient may be retrieved from the coordinates. For another example, as for body posture, orientations of a plurality of body parts (e.g. upper arms, lower arms, laps, and calves) of the patient may be acquired by an angle sensor (e.g., a gyroscope sensor), wherein the body posture of the patient may be retrieved from the orientations. For yet another example, the physiological indicator of body temperature, heart rate, pulse, blood pressure, blood oxygen saturation, complexion, or skin smoothness of the patient may be acquired by a specialized sensor (e.g. a body temperature sensor, a heart rate sensor, a pulse sensor, a blood pressure sensor, a blood oxygen saturation sensor, a binocular camera, or a smell sensor). For yet another example, a smart stethoscope may be used to acquired sounds from the patient's body (e.g. sounds from organs such as the heart, the liver, the lungs, sound of pulses, sound from blood vessels). Here, the smart stethoscope is a new kind of stethoscope that does not require a physical connection between the operator's ears and the head of the stethoscope. Instead, the audio may be transmitted via Bluetooth or other wireless communication channels.

In some embodiments, the at least one sensor may be integrated into a wearable device. For example, various categories of sensors may be integrated into a smartwatch, to measure the patient's body temperature, heart rate, pulse, blood pressure, and blood oxygen saturation at the same time. For another example, a position sensor and a pressure sensor may be integrated into a wearable sensory overall.

The physical examination device 110 may also be configured to perform a physical examination operation (e.g., a second target examination operation) on the patient based on examination information sent from the first extended reality device 120 at the operator end.

In some embodiments, the physical examination device 110 may include a first wearable sensory device, which can perform a physical examination operation on the patient based on the examination information. For example, at least one touch emulating device may be placed on one or more locations on the first wearable sensory device, to emulate the touches the patient feels when receiving a physical examination operation. The at least one touch emulating device may include a vibration component (e.g., a vibrating motor), a microcurrent stimulation component, an airbag component, or the like, or any combination thereof. The microcurrent stimulation component may have a stimulation intensity within a safe and healthy range (e.g., 0-500 μA) for the patient. The airbag component may emulate touches (e.g., the feeling of being pressed on the skin by a finger) by adjusting a contact area and an internal pressure of one or more airbags. As aforementioned, at least one sensor may also be placed on one or more locations on the first wearable sensory device, and the at least one sensor may collect the physical information from the patient. In some embodiments, a touch emulating device and a sensor at the same location on the first wearable sensory device may be integrated.

The present disclosure does not intend to limit how a wearable sensory device (e.g., the first wearable sensory device and/or a second wearable sensory device that will be mentioned below) is designed and implemented. In some embodiments, the wearable sensory device may be a tight overall. The overall may cover the entirety or a part of the patient's body. Therefore, the overall is convenient for full-body or half-body examinations. In some embodiments, the wearable sensory device may be specialized for localized physical examination, e.g., examination for the head, hand, foot, knee, etc. In these scenarios, the wearable sensory device may be designed as a device that can cover one body part only. For example, the wearable sensory device may be in the form of a helmet, a hat, a sock, a glove, a boot, or a knee brace.

In some embodiments, the first wearable sensory device (e.g. an overall) may also be configured to assist the patient with posing or performing certain actions for clinical purposes (e.g. treatment for benign paroxysmal positional vertigo). For example, the position sensor and/or the angle sensor in the first wearable sensory device may check the patient's posture. The motion sensor (e.g., an acceleration sensor) may check if the patient is moving or stationary. In some embodiments, when sensing that there is a discrepancy between the patient's posture and a reference posture for treatment or examination, the physical examination device 110 may assist the patient in correcting his/her posture based on the discrepancy. For example, the reference posture may be raising an arm to a reference height. If the patient's arm fails to meet the reference height, the airbag component on the patient's arm may apply an upward pressure to help the patient raise his/her arm. When the patient's arm reaches the reference height, the physical examination device 110 may send a reminder to the patient. For example, the vibration component or the microcurrent stimulation component in the arm position of the first wearable sensory device may be controlled to output a prompt signal (e.g., in the form of vibration or current) to indicate that the patient has moved to a target position.

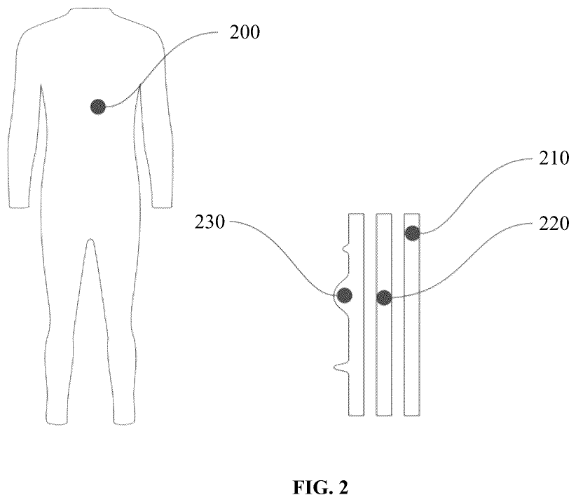

FIG. 2 is a schematic diagram illustrating a front view and a cross-sectional view of an exemplary wearable sensory overall according to some embodiments of the present disclosure.

As shown in FIG. 2, the wearable sensory overall may be multi-layered. A vibration component 210, a microcurrent stimulation component 220, and an airbag component 230 may be respectively embedded in three layers. The airbag component 230 may be embedded in the innermost layer, so that the airbag component 230 may be in direct contact with the surface of the patient's body.

To ensure the effective transmission of touch emulation and the accuracy of body measurements, the wearable sensory overall may be made from highly elastic materials, which can be worn tightly on the body of the patient.

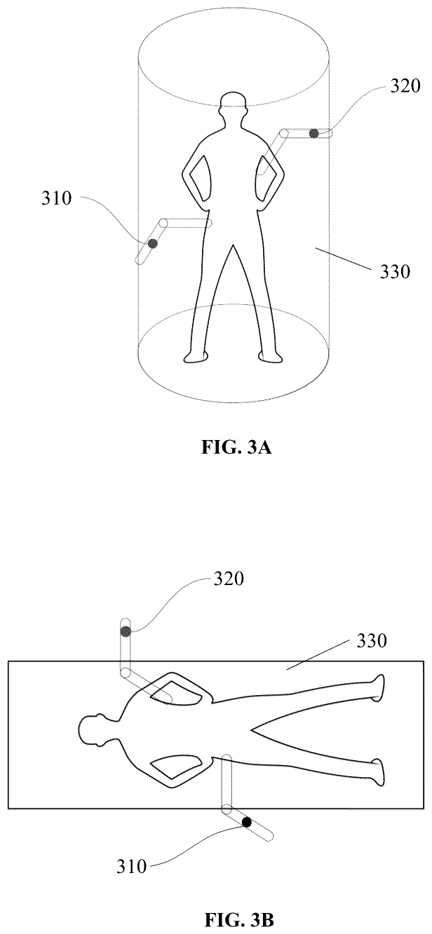

In some embodiments, the physical examination device 110 may include one or more mechanical arms configured to perform a physical examination operation on the patient based on the examination information. In some embodiments, an operation end of the mechanical arm may resemble a human hand to perform a more realistic physical examination operation. Merely by way of example, according to FIGS. 3A and 3B, the physical examination device 110 may include a first mechanical arm 310 and a second mechanical arm 320. The two mechanical arms may simulate a face-to-face physical examination performed by an operator with one hand or both hands on a patient.

In some embodiments, the patient end may include a positioning device configured to determine positioning information (e.g., locations of a plurality of points and/or regions) on the surface of the patient's body. The positioning information may be used to generate the digital twin, perform a physical examination operation, coordinate conversion, etc. As aforementioned, in some embodiments, the position sensor on the first wearable sensory device may locate a plurality of points on the surface of the patient's body. In other words, the first wearable sensory device has a positioning function (e.g., the first wearable sensory device may be regarded as a positioning device). In some embodiments, as shown in FIGS. 3A and 3B, the positioning function may be realized by applying air currents or ultrasounds in a sensory space 330. The air currents may be emitted by an air current positioning device. The ultrasounds may be emitted by an ultrasonic positioning device. The positioning device (e.g., the air current positioning device or the ultrasonic positioning device) may be placed near the patient's body (e.g., at a certain distance from the patient). The positioning device may emit detecting waves (ultrasounds or air currents) and detect reflected waves or scattered waves back from the patient's body surface, thereby positioning points and/or regions on the patient's body surface. According to FIG. 3A, when a patient receives physical examination in a standing pose in the space 330, the positioning device may be placed around the patient's body. According to FIG. 3B, when a patient receives physical examination while lying on a bed in the space 330, the positioning device may be placed above the patient's body.

The first extended reality device 120 may obtain the physical information of the patient, generate a digital twin of the patient based on the physical information, and display the digital twin.

Herein, extended reality is used as a collective name for virtual reality (VR), augmented reality (AR), and mixed reality (MR).

VR uses computer technology to create a virtual scene. A user can use a head-mounted display to observe virtual objects in a virtual scene, and can use other peripherals (also called accessories) to interact with the virtual objects. For example, users can use VR glasses to observe virtual user interfaces, virtual characters, etc. in virtual scenes, and use VR handles to perform an input operation on the user interface, or interact with virtual characters, etc.

AR senses the real world through computer technology, generates a virtual information layer, and superimposes the virtual information layer onto the real background visible to the naked eyes (e.g., displaying vehicle speed and navigation information on a front windshield of a vehicle), thus completing a combination of reality and virtuality.

MR uses computer technology to mix the real world and a virtual scene together, allowing interaction between the virtual scene and the real world. For example, virtual characters will detour when encountering real obstacles. Similar to VR, users can see real objects (such as real houses) and virtual objects (such as virtual user interfaces, or virtual characters, etc.) in a mixed scene through an MR head-mounted display.

Via the first extended reality device 120, the operator may see the patient's digital twin and interact with the digital twin. Based on the actual technology used (VR/AR/MR), the display of the digital twin and/or interaction with the digital twin may be realized through the second external device of the first extended reality device 120 (such as a head-mounted display or an interactive handle), or without the second external device (e.g., visible to the naked eyes, or interacting with a projection of the digital twin using hands).

In some embodiments, the first extended reality device 120 may include a touch emulating device configured to emulate the touch the operator feels when performing a first target examination operation. The operator may perform a first target examination operation on the digital twin through the touch emulating device and get intuitive feedback from the emulated touch senses. The touch emulating device may be an interactive external device of the first extended reality device 120 (e.g., VR or MR devices) on the operator end. The operator may perform a first target physical examination operation on the digital twin in a virtual scene via the touch emulating device, and the first extended reality device 120 may capture the first target examination operation and generate the corresponding examination information.

A touch emulating component (e.g. an airbag component) and a sensor (e.g. a position sensor and/or a gyroscope sensor) may be placed at one or more locations on the touch emulating device. Detailed descriptions regarding the touch emulating component and the sensor can be found in the section describing the wearable sensory device.

The present disclosure does not intend to limit the form of the touch emulating device. For example, the touch emulating device may include a touch emulating glove. For another example, the touch emulating device may include one or more touch emulating finger cots. As yet another example, the touch emulating device may include one or more touch emulating stickers attached to the pad of at least one finger.

In some embodiments, the system 100 may also include a second extended reality device 140 on the patient end. Details regarding the second extended reality device 140 may refer to the section describing the first extended reality device 120. The first extended reality device 120 and the second extended reality device 140 may be in communication so that the patient and the operator can communicate remotely via the first extended reality device 120 and the second extended reality device 140. For example, the operator and the patient may set up voice or video calls via the first extended reality device 120 and the second extended reality device 140. For another example, the operator and the patient may communicate via text messages via the first extended reality device 120 and the second extended reality device 140.

In some embodiments, the patient may request an instruction of using the physical examination device 110 by inputting voice or text into the second extended reality device 140, e.g., a wearing guidance of the first wearable sensory device, or a wearing guidance of a wearable device in which various sensors are integrated. The second extended reality device 140 may provide the instruction (e.g., in the form of voice, video, or text, etc.) of using the physical examination device 110 based on the patient's input. When the patient uses the physical examination device 110 incorrectly, the second extended reality device 140 may generate a reminder for the patient.

In some embodiments, the first extended reality system 120 may include a second wearable sensory device worn by the operator, a third party, or a human body model. For example, the second wearable sensory device may be a tight overall. At least one sensor may be placed at one or more locations on the second wearable sensory device. In some embodiments, the at least one sensor may include a position sensor and a pressure sensor, to detect an examination position (e.g., represented by spatial coordinates) on the second wearable sensory device and a force (e.g., including a direction and/or an intensity of the force) applied to the examination position. The operator may perform a first target examination operation (P1) at examination position Q1 of the second wearable sensory device. The second controller of the first extended reality device 120 may generate the examination information and send the examination information to the first controller of the physical examination device 110. Based on the examination information, the physical examination device 110 on the patient end may perform a second target examination operation P2 on the patient through the first wearable sensory device. The examination position of the second target examination operation performed by the first wearable sensory device may be noted as Q2. Since the object wearing the second wearable sensory device and the patient may have different body sizes, Q1 needs to be converted to Q2 based on the object's body sizes and the patient's body sizes, e.g., based on a ratio between the object's body sizes and the patient's body sizes.

In some embodiments, the system 100 may also include an imaging device 150 (e.g., a 360-degree panoramic camera, a 3D camera, etc.) on the patient end. The imaging device 150 may capture real-time images (e.g., two-dimensional (2D) or 3D) of the patient and send the real-time images to the first extended reality device 120. The first extended reality device 120 may display the real-time images.

Compared to the patient's real-time images, the digital twin of the patient may focus on displaying the patient's “hidden” physiological indicators in real-time, where these physiological indicators may not be visible or hardly visible to the naked eyes. For example, the digital twin may display the values of the patient's heart rate, temperature, pulse, blood pressure, blood oxygen saturation, smell, etc., in real-time.

Given the fact that there should be no significant discrepancies between the patient's real-time images and the digital twin, when the operator observes major discrepancies between the patient's real-time images and the digital twin, it may be an indicator that the system 100 is malfunctioning (e.g. unstable network connections, a mistake in the generation of the digital twin). In those events, certain measures may be taken to ensure the normal functioning of the remote physical examination, such as improving the quality of the network connections, and/or regenerating a digital twin.

When the operator and the patient communicate through the first extended reality device 120 and the second extended reality device 140, communication through voice will create barriers for patients with hearing or language impairments, or, if the patient has an accent, there will be barriers to communication with the operator. In response to the above problems, the second extended reality device 140 may provide a function of displaying subtitles and/or sign language. The patient and the operator may choose whether to turn on this function. The first extended reality device 120 and/or the second extended reality device 140 may use a voice recognition model to convert the voice of the patient and/or the operator into subtitles for display. The first extended reality device 120 and/or the second extended reality device 140 may use a sign language generation model to convert the operator's voice into sign language for display, and/or use the sign language generation model to convert the patient's sign language to voice or subtitles for display. This function makes the communication between the operator and the patient smoother and the remote physical examination more efficient.

In some embodiments, the first extended enhancement device 120 may use automatic conference summarization technology or a language model to automatically generate a diagnostic report based on the communication between the operator and the patient during the remote physical examination. The diagnostic report may include the patient's basic information, a symptom described by the patient, a diagnosis result determined by the operator, the operator's treatment recommendation, or the like, or any combination thereof. The operator may not have time to record a diagnosis process at the same time during the physical examination.

Automatically generating a diagnostic report based on the communication between the operator and the patient during the physical examination may reduce the operator's workload and pressure.

The network 130 may be used to facilitate transmission of information and/or data between the patient end and the operator end. For example, the physical examination device 110 and/or the imaging device 150 may send the patient's physical information to the first extended reality device 120 via the network 130. For another example, the first extended reality device 120 may send the examination information to the physical examination device 110 via the network 130. For yet another example, the first extended reality device 120 on the patient end and the second extended reality device 140 on the operator end may communicate via the network 130.

For privacy purposes, the physical examination, as well as collection, storage, and usage of personal information (e.g. physical information) must be performed with the patient's consent. In some embodiments, the patient may adjust privacy settings via the second extended reality device 140. For example, for certain body parts, the patient may deny the operator's access for physical examination and physical information collection. In some embodiments, the operator may request, through the first extended reality device 120, access to the second extended reality device 140. Correspondingly, the patient may grant or deny access through the second extended reality device 140.

For safety purposes, the system 100 may include a safety module. For example, the force output by the wearable sensory device or the mechanical arm may be limited below a safety threshold. For another example, the current output by the microcurrent stimulation component may also be limited below a safety threshold. For yet another example, the patient may gradually experience different levels of force or current outputs from the system 100 from the lowest level above, until the patient confirms that the current experienced force or current is the maximum level he/she could handle.

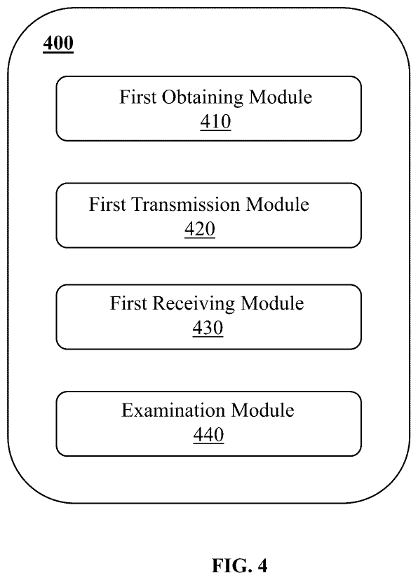

FIG. 4 is a schematic block diagram illustrating an exemplary system for remote physical examination implemented on a patient end according to some embodiments of the present disclosure.

The system 400 may be implemented on the physical examination device 110 through hardware and/or software components. As shown in FIG. 4, the system 400 on the patient end may include a first obtaining module 410, a first transmission module 420, a first receiving module 430, and an examination module 440.

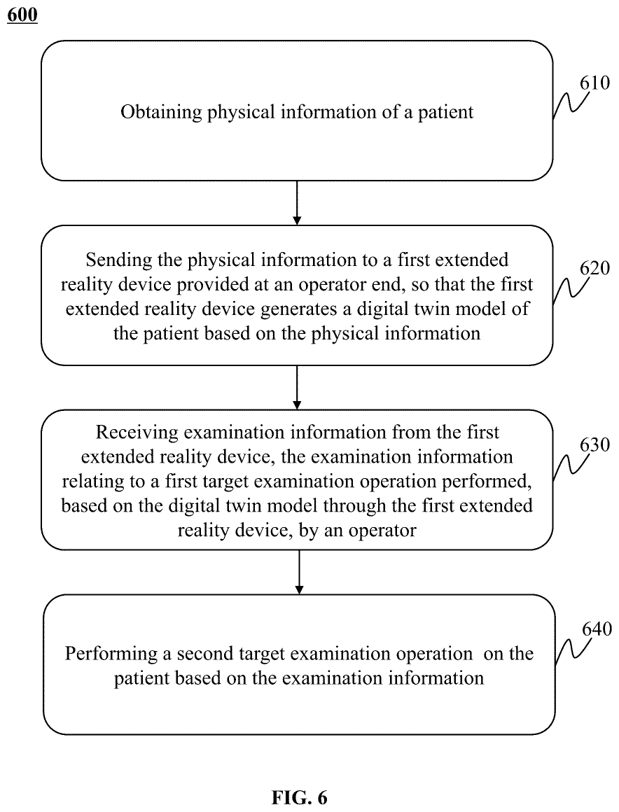

The first obtaining module 410 may collect the patient's physical information. Details may be found in operation 610 of process 600 in FIG. 6.

The first transmission module 420 may send the physical information to the first extended reality device 120 so that the first extended reality device 120 generates a digital twin of the patient based on the physical information. Details may be found in operation 620 of the process 600 in FIG. 6.

The first receiving module 430 may receive examination information from the first extended reality device 120. Details may be found in operation 630 of the process 600 in FIG. 6.

The examination module 440 may perform a second target examination operation on the patient based on the examination information. Details may be found in operation 640 of the process 600 in FIG. 6.

FIG. 5 is a schematic block diagram illustrating an exemplary system for remote physical examination implemented on an operator end according to some embodiments of the present disclosure. The system 500 may be implemented on the first extended reality device 120 through hardware and/or software components.

As shown in FIG. 5, the system 500 may include a second receiving module 510, a modeling module 520, a display module 530, a third receiving module 540, and a second transmission module 550.

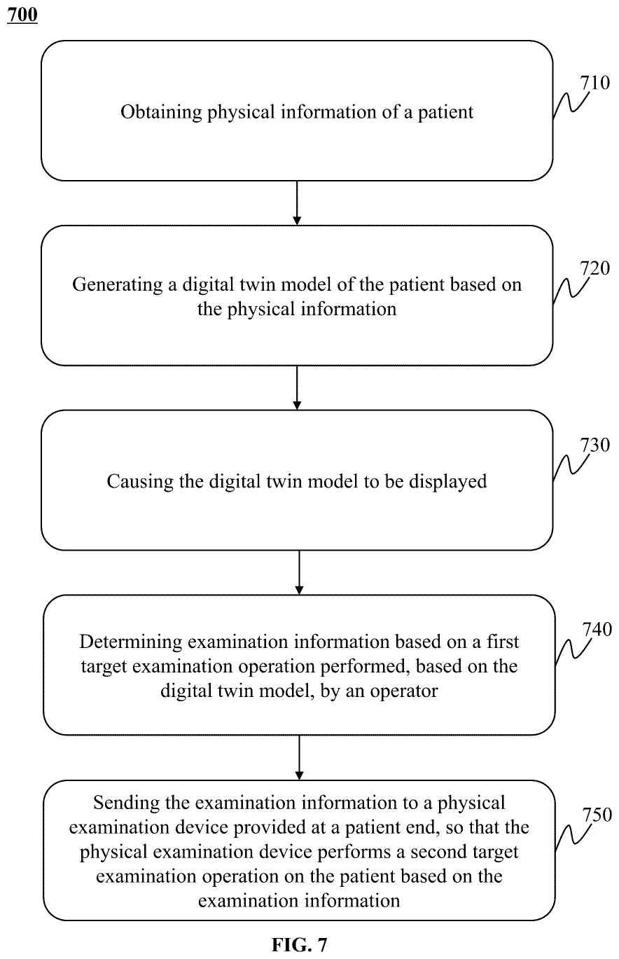

The second receiving module 510 may obtain the physical information of the patient. Details may be found in operation 710 of process 700 in FIG. 7.

The modeling module 520 may generate a digital twin of the patient based on the physical information. Details may be found in operation 720 of the process 700 in FIG. 7.

The display module 530 may display the digital twin. Details may be found in operation 730 of the process 700 in FIG. 7.

The third receiving module 540 may determine examination information related to a first target examination operation performed by the operator. Details may be found in operation 740 of the process 700 in FIG. 7.

The second transmission module 550 may send the examination information to the physical examination device 110. Details may be found in operation 750 of the process 700 in FIG. 7.

It should be noted that the systems and modules shown in FIGS. 4 and 5 can be realized in various ways. For example, the system and its modules can be realized through software, hardware, and/or a combination of software and hardware. For example, the hardware part may be one or more integrated circuits designed for specific purposes, and the software part may be stored in one or more storage devices, and executed by one or more execution systems, such as a microcontroller or a specifically designed hardware device.

It should be noted that the descriptions of the systems and modules above are merely for convenience and do not intend to constitute limitations of the scope of the present disclosure. For a person with ordinary skills in the art, as they understand the working principle of the system, they may connect and combine the modules, devices, and sub-systems in various ways without deviating from the working principle. For example, the first obtaining module 410 and the first output module may either be distinct modules or combined as one single module. For another example, the third receiving module 540 and the second transmission module 550 may either be distinct modules or combined as one single module. Such variations of the system are also within the scope of the present disclosure.

FIG. 6 is a flowchart illustrating an exemplary process for remote physical examination implemented on a patient end according to some embodiments of the present disclosure. In some embodiments, the process 600 may be implemented in the system 100 illustrated in FIG. 1. For example, the process 600 may be stored in a storage device as a form of instructions, and can be invoked and/or executed by the physical examination device 110 (e.g., the first controller of the physical examination device 110, or one or more modules illustrated in FIG. 4). The operations of the illustrated process 600 presented below are intended to be illustrative. In some embodiments, the process 600 may be accomplished with one or more additional operations not described, and/or without one or more of the operations discussed. Additionally, the order in which the operations of the process 600 as illustrated in FIG. 6 and described below is not intended to be limiting.

In 610, the physical examination device 110 (e.g., the first obtaining module 410) may obtain physical information of a patient.

More information regarding how to obtain the physical information may be found elsewhere in the present disclosure (e.g., in connection with FIG. 1). For example, the first obtaining module 410 may include at least one sensor configured to acquire the physical information as illustrated in FIG. 1. As another example, the first obtaining module 410 may obtain the physical information from at least one sensor illustrated in FIG. 1.

In 620, the physical examination device 110 (e.g., the first transmission module 420) may send the physical information to the first extended reality device 120, so that the first extended reality device 120 generates a digital twin of the patient based on the physical information. Details regarding generating the digital twin by the first extended reality device 120 may be found elsewhere in the present disclosure (e.g., in connection with FIG. 7).

In 630, the physical examination device 110 (e.g., the first receiving module 430) may receive examination information from the first extended reality device 120. The examination information may relate to a first target examination operation performed, based on the digital twin through the first extended reality device 120, by an operator (e.g., a doctor). Details regarding determining the examination information by the first extended reality device 120 may be found elsewhere in the present disclosure (e.g., in connection with FIG. 7, in particular, operation 740 of process 700 in FIG. 7).

In 640, the physical examination device 110 (e.g., the examination module 440) may perform a second target examination operation on the patient based on the examination information. In some embodiments, the first target examination operation may be the same as or different from the second target examination operation. In some embodiments, the examination module 440 may include a first wearable sensory device and/or at least one mechanical arm.

In some embodiments, the examination information may represent the first target examination operation, and include at least one examination position of the first target examination operation and at least one of an examination type, an examination direction, or a first intensity of the first target examination operation. In some embodiments, the examination position may include a label indicating a body part and/or spatial coordinates of a position on the body surface of the patient. For example, if the remote physical examination requires a lower level of spatial accuracy, the examination position in the examination information may be a label of the body part. For another example, if the remote physical examination requires a high level of spatial accuracy, the examination position in the examination information may include spatial coordinates of a position on the body surface of the patient. In some embodiments, the examination type may include one or more of a press, stroking, or a tap. In some embodiments, the first intensity of the first target examination operation may be represented by a level (e.g., low/medium/high). In some embodiments, the first intensity of the first target examination operation may be represented by a value. In some embodiments, the examination direction of the first target examination operation refers to a direction of force applied to the at least one examination position in the first target examination operation. It's to be noted that the examination direction may be omitted in the examination information. For example, the system 100 may automatically determine that the examination direction is vertically to the body surface of the corresponding examination position.

In some embodiments, the physical examination device 110 may restore the first target examination operation to the patient based on the examination information. Restoring the first target examination operation to the patient means that the physical examination device 110 may perform a second target examination operation same as the first target examination operation. For example, if the examination information includes “right knee” (the examination position) and “tap” (the examination type), the second target examination operation may be a tap of the right knee. As another example, the examination information may be represented as a group of parameters characterizing the first target examination operation, such as (S1, L1, F1; S2, L2, F2), wherein S1 represents a first examination position, L1 represents a first examination direction of force applied to the first examination position S1, F1 represents a value of the intensity of the force applied to the first examination position S1, S2 represents a second examination position, L2 represents a second examination direction of force applied to the second examination position S2, and F2 represents a value of the intensity of the force applied to the second examination position S2. The physical examination device 110 may generate, based on the examination information, the parameters characterizing the first target examination operation, and restore the first target examination operation to the patient based on the parameters, e.g., the physical examination device 110 may control, based on the parameters, the first wearable sensory device and/or the at least one mechanical arm to move to restore the first target examination operation to the patient.

In some embodiments, the physical examination device 110 may control the airbag component located at the first examination position S1 on the first wearable sensory device to apply a force with the first examination direction L1 and the value F1, and control the airbag component located at the second examination position S2 on the first wearable sensory device to apply a force with the second examination direction L2 and the value F2. In some embodiments, the physical examination device 110 may control the mechanical arm to apply, to the first examination position S1 on the body surface, a force with the first examination direction L1 and the value F1, and apply, to the second examination position S2 on the body surface, a force with the second examination direction L2 and the value F2.

In some embodiments, the ultrasonic positioning device and the air current positioning device may also have a touch emulating function. Correspondingly, the ultrasonic positioning device and/or the air current positioning device may also be used to perform the second target examination operation on the patient. Like air currents, ultrasounds can also be felt by the human body. After placing the ultrasonic positioning device and/or the air current positioning device in an enclosed environment (such as an enclosed indoor space), accurate positioning on the patient's body surface and touch emulation may be achieved by adjusting parameters of emitted waves from the ultrasonic positioning device and/or the air current positioning device. Taking the air current positioning device as an example, the air current positioning device may include an array of air outlets, and the physical examination device 110 may control parameters of the air currents output by the air outlets, such as, a direction of the air currents, an intensity (velocity) of the air currents, a shape of the air outlet, and duration of outputting the air currents, to realize accurate positioning on the patient's body surface and touch emulation. Since the positioning device (e.g., the ultrasonic positioning device and/or the air current positioning device) has a positioning function, the positioning device may accurately reconstruct the examination position to perform the second target examination operation. Also, same as the example in the last paragraph, when the examination information is (S1, L1, F1; S2, L2, F2), the physical examination device 110 may adjust the parameters of the emitted waves (e.g., ultrasounds or air currents), so that a force with a first direction L1 and a value F1 is applied to the first examination position S1 on the patient's body surface, and a force with a second direction L2 and a value F2 at the second examination position S2 on the patient's body surface.

When the operator performs the first target examination operation through the first extended reality device 120, since the first target examination operation does not directly act on the patient, the force of the first target examination operation may be too large. For the patient's injury, or a position that is sensitive to force, if the force of the first target examination operation is too great, mapping the first target examination operation to the mechanical arm or the first wearable sensory device may cause secondary damage or discomfort to the patient.

Aiming at the above problem, the physical examination device 110 may determine whether the first intensity is within a protection range. In response to determining that the first intensity is outside the protection range, the physical examination device 110 may determine a second intensity based on the protection range. The physical examination device 110 may perform the second target examination operation on the patient based on the second intensity. In response to determining that the first intensity is within the protection range, the physical examination device 110 may restore the first target examination operation to the patient based on the examination information, e.g., the physical examination device 110 may perform a second target examination operation same as the first target examination operation.

In some embodiments, the protection range may be determined using a protection range determination model based on at least one of patient information or medical records of the patient. The protection range determination model may be a machine learning model. The input of the protection range determination model may include at least one of patient information or medical records of the patient. The output of the protection range determination model may include the protection range. In some embodiments, a patient may correspond to a single protection range. In some embodiments, different parts of the patient may correspond to different protection range. For example, legs of the patient may correspond to a first protection range, and breast of the patient may correspond to a second protection range.

The patient information of the patient may include the age, gender, occupation, or the like, or any combination thereof. The medical records of the patient may include a type of the disease, a location of the disease, one or more medical images of the patient, treatment information of the disease, or the like, or any combination thereof.

In some embodiments, the protection range determination model may be provided by training a preliminary model using sample data. The sample data may include a plurality of sample data sets. Each of the plurality of sample data sets may include training data and label data of a sample patient. The training data of the sample patient may include patient information and/or medical records related to a face-to-face physical examination of the sample patient. The label data of the sample patient may include a protection range of the sample patient. The protection range of the sample patient may be obtained based on feedback of the sample patient in the face-to-face physical examination.

In some embodiments, in response to determining that the first intensity is smaller than a minimum value of the protection range, the physical examination device 110 may determine the minimum value of the protection range as the second intensity. In response to determining that the first intensity is greater than a maximum value of the protection range, the physical examination device 110 may determine the maximum value of the protection range as the second intensity.

In some embodiments, the process for adjusting the first intensity to the second intensity based on the protection range may be performed by the first extended reality device 120. In this case, the examination information may include at least one examination position and at least one of the examination type, the examination direction, and the second intensity. The physical examination device 110 may perform the second target examination operation on the patient directly according to the examination information. The process for adjusting the first intensity to the second intensity based on the protection range performed by the first extended reality device 120 may be similar to the above process for adjusting the first intensity to the second intensity based on the protection range performed by the physical examination device 110.

In some embodiments, after the physical examination device 110 performs the second target examination operation on the patient, the physical examination device 110 may record feedback (e.g., video, voice, text, etc.) of the patient on the second target examination operation, and adjust the protection range based on the patient's feedback. For example, after the physical examination device 110 performs the second target examination operation on the patient, the patient expressed that he/she could not bear the force of the second target examination operation. The physical examination device 110 may identify the voice of the expression of the patient and decrease the maximum value of the protection value.

In some embodiments, spatial coordinates of the examination position of the first target examination operation performed through the first extended reality device 120 (e.g., the second wearable sensory device or the digital twin) may be converted to spatial coordinates of the corresponding position on the patient. The coordinate conversion may be performed by the first extended reality device 120 or the physical examination device 110. A conversion relationship may be determined based on a coordinate system of the first extended reality device 120 and a coordinate system of the physical examination device 110. Moreover, the conversion relationship may be determined based on a ratio between a size of the digital twin and a size of the patient's body, or a ratio between a size of an object wearing the second wearable sensory device and a size of the patient's body.

It should be noted that the above description is merely provided for the purposes of illustration, and not intended to limit the scope of the present disclosure. For persons having ordinary skills in the art, multiple variations and modifications may be made under the teachings of the present disclosure. However, those variations and modifications do not depart from the scope of the present disclosure.

FIG. 7 is a flowchart illustrating an exemplary process for remote physical examination implemented on operator end according to some embodiments of the present disclosure. In some embodiments, the process 700 may be implemented in the system 100 illustrated in FIG. 1. For example, the process 700 may be stored in a storage device as a form of instructions, and can be invoked and/or executed by the first extended reality device 120 (e.g., the second controller of the first extended reality device 120, or one or more modules illustrated in FIG. 5). The operations of the illustrated process 700 presented below are intended to be illustrative. In some embodiments, the process 700 may be accomplished with one or more additional operations not described, and/or without one or more of the operations discussed. Additionally, the order in which the operations of the process 700 as illustrated in FIG. 7 and described below is not intended to be limiting.

In 710, the first extended reality device 120 (e.g., the second receiving module 510) may obtain physical information of a patient.

Details regarding the physical information may be found elsewhere in the present disclosure (e.g., in connection with FIG. 1 and FIG. 6). In some embodiments, at least a portion of the physical information may be obtained from the patient end. For example, when the patient comes for a follow-up visit, the first extended reality device 120 may retrieve static physical information of the patient from medical records of the patient. Or the patient may let the operator know his/her age, gender, and body sizes in a conversation. The static physical information refers to physical information that stays constant for a long time (such as within a year) or at least stays constant during the period of a visit. The static physical information may include age, gender, body sizes, or the like, or any combination thereof.

In 720, the first extended reality device 120 (e.g., the modeling module 520) may generate a digital twin of the patient based on the physical information.

In 730, the first extended reality device 120 (e.g., the display module 530) may cause the digital twin model to be displayed. In some embodiments, the first extended reality device 120 may cause the digital twin model and real-time images (e.g., 2D or 3D) of the patient to be displayed together or individually.

The digital twin may include a three-dimensional (3D) body model and annotated information.

The 3D body model may be generated based on body sizes of the patient and/or a real-time video of the patient. The 3D body model may include a body surface. The body surface may refer to a naked part (such as skin, nails) and a semi-naked part (such as nostrils) of a human body. In some embodiments, the 3D body model may also include an internal structure of a human body (such as bones, organs, tissue, and blood vessels). In some embodiments, the first extended reality device 120 may calibrate a reference human body model based on the body size and/or the real-time video of the patient to get the 3D body model of the patient.

The annotated information may include the physical information of the patient. For example, the first extended reality device 120 may label the heart rate of the patient on a region of heart of the 3D body model. For another example, the first extended reality device 120 may label the skin color and smoothness on a target skin region of the 3D body model. As still another example, the first extended reality device 120 may label the current blood pressure of the patient on the 3D body model. In some embodiments, the annotated information may also include data from the patient's past diagnosis. For example, the first extended reality device 120 may label a position and/or a type of an abnormality (e.g., an injury, a tumor, etc.) of the patient. The annotated information may provide a reference for the operator to perform the first target examination operation.

In some embodiments, the operator may zoom in or out of the 3D body model. Understandably, the zooming in/out only changes the size but not the shape of the 3D body model.

In some embodiments, the first extended reality device 120 may include an audio output device (such as earphones). The audio output device may play the sounds collected from the patient's body (e.g. heart sound, pulmonary sound, etc.) from a smart stethoscope on the patient end for the operator's reference.

In some embodiments, the first extended reality device 120 may generate a real-time digital twin of the patient based on the physical information, and cause the digital twin model to be displayed in real time. For example, if the patient makes a movement (e.g., a posture and/or a location of the patient changes), the digital twin may make a corresponding movement. As another example, the annotated information (e.g., the physical information of the patient) of the digital twin may be displayed in real-time. In some embodiments, the digital twin may present a fixed posture and/or location of the patient, which does not change as the patient moves. For example, the first extended reality device 120 may reconstruct a 3D body model of the digital twin based on at least one video frame in which the patient is in a standing posture, resulting in a digital twin in a standing posture. When the patient sits on a chair, the digital twin may still keep the standing posture.

In some embodiments, the digital twin may include visualization of at least one diseased part of the patient. The at least one diseased part may include at least one of a suspected diseased part or a definite diseased part.

The first extended reality device 120 may reconstruct the suspected diseased part based on the medical records, the real-time video, one or more medical images of the patient, or the like, or any combination thereof.

The suspected diseased part may be predicted using a diseased part prediction model based on at least one of patient information or medical records of the patient. The diseased part prediction model may be a machine learning model. The input of the diseased part prediction model may include at least one of patient information or medical records of the patient. The output of the diseased part prediction model may include the suspected diseased part.

In some embodiments, the diseased part prediction model may be provided by training a preliminary model using sample data. The sample data may include a plurality of sample data sets. Each of the plurality of sample data sets may include training data and label data of a sample patient. The training data of the sample patient may include patient information and/or medical records related to a doctor visit of the sample patient. The label data of the sample patient may include a diseased part determined in the subsequent visit rather than the current doctor visit.

Through the digital twin, the operator may directly view the suspected diseased part and/or the definite diseased part, so that the operator can more accurately make a diagnosis for the patient, and refer to the suspected diseased part and/or the definite diseased part to perform a more appropriate first target examination operation.

In 740, the first extended reality device 120 (e.g., the third receiving module 540) may determine examination information based on a first target examination operation performed, based on the digital twin, by the operator.

In some embodiments, the operator may use a touch emulating device (such as a touch emulating glove) to perform a first target examination operation by touching the digital twin or the real-time images, and the first extended reality device 120 may sense the examination position and at least one of an examination type, an examination direction, and a first intensity of the first target examination operation. In some embodiments, the first extended reality device 120 may determine at least one examination position and at least one of the examination type, the examination direction, and the first intensity of the first target examination operation as the examination information. For the purpose of illustration, description related to performing the first target examination operation may take the digital twin as an example. The operation of performing the first target examination operation on the real-time images may be similar to the operation of performing the first target examination operation the digital twin.

In some embodiments, in response to detecting that the touch emulating device touches the digital twin (e.g., a point on the touch emulating device overlaps any point on the digital twin), the first extended reality device 120 (e.g., a VR device, or an MR device) may ask for a confirmation from the operator regarding whether to generate the examination information. In response determining that the operator confirms that the examination information needs to be generated, the first extended reality device 120 may further ask for a confirmation about details of the examination information to be generated. For example, the first extended reality device 120 may ask for a confirmation regarding whether to generate the type, the direction, or the intensity of the first target examination operation. The first extended reality device 120 may obtain the confirmation of the patient through a voice input, a video input, and/or a text input of the patient. In some embodiments, the operator may input at least a part of the examination information (e.g., at least one of the examination position, the examination type, the examination direction, and the examination intensity) by voice or text. In some embodiments, the operator may touch the digital twin by one hand to determine at least one examination position and input at least one of the examination type, the examination direction, and the first intensity by another hand. In some embodiments, in response determining that the operator confirms that the examination information needs to be generated, the first extended reality device 120 may directly generate the examination information. Information regarding whether to generate the type, the direction, or the intensity of the first target examination operation and/or which of the type, the direction, or the intensity of the first target examination operation required to be included in the examination information may be default of the first extended reality device 120 or set by the operator in advance. In some embodiments, in response to detecting that the touch emulating device touches the digital twin, the first extended reality device 120 may directly generate the examination information. Information regarding whether to generate the type, the direction, or the intensity of the first target examination operation and/or which of the type, the direction, or the intensity of the first target examination operation required to be included in the examination information may be default of the first extended reality device 120 or set by the operator in advance.

In some embodiments, the touch emulating device (e.g., a touch emulating glove) may output a force back to the operator, and the force may be with a same value as the first intensity, so that the operator may confirm if the input first intensity is valid. When the operator confirms that the input first intensity is valid, the first extended reality device 120 may generate the examination information based on at least one examination position at which the touch emulating device touches, and the first intensity confirmed as valid by the operator.

In some embodiments, the first extended reality device 120 may use the examination information to emulate, through the touch emulating device, a touch the operator feels when performing the first target examination operation on the patient. For example, the touch emulating device may output, to the operator, a force with an intensity equal to the first intensity and a direction opposite to the examination direction.

In some embodiments, in response to detecting that the operator's hand touches the digital twin (e.g. a point on the hand overlaps any point on the digital twin), the first extended reality device 120 (e.g., an MR device) may ask for a confirmation from the operator regarding whether to generate examination information. In response to determining that the operator confirms to generate the examination information, the first extended reality device 120 may ask for a confirmation regarding a value of a force to be applied. The method to confirm the value of the force may include obtaining a value input by the operator. In some embodiments, the operator may input the value of the force by an audio input, or the operator may use one hand to select at least one examination position and use another hand to input the value of the force. In some embodiments, the first extended reality device 120 may determine the examination direction of the force by detecting the operator's hand posture.

In some embodiments, in response to detecting that the operator performs a first target examination operation on the second wearable sensory device (e.g., a wearable sensory overall), the corresponding examination information may be obtained. The second wearable sensory device may capture the first target examination operation and generate the corresponding examination information. More details about capturing the first target examination operation via the second wearable sensory device may be found in the descriptions of FIG. 1.

In some embodiments, the operator may directly input the examination information without performing the first target examination operation. For example, the operator may input the examination information manually or by voice.

In some embodiments, the process for adjusting the first intensity to the second intensity based on the protection range may be performed by the first extended reality device 120. In this case, the examination information may include at least one examination position and at least one of the examination type, the examination direction, and the second intensity. The process for adjusting the first intensity to the second intensity based on the protection range performed by the first extended reality device 120 may be similar to the process for adjusting the first intensity to the second intensity based on the protection range performed by the physical examination device 110 (e.g., as illustrated in FIG. 6, in particular, operation 640 of the process 600 in FIG. 6).

In 750, the first extended reality device 120 (e.g., the second transmission module 550) may send the examination information to the physical examination device 110, so that the physical examination device 110 performs a second target examination operation on the patient based on the examination information. Details regarding performing the second target examination operation on the patient based on the examination information by the physical examination device 110 may be found elsewhere in the present disclosure (e.g., in connection with FIG. 6, in particular, operation 640 of the process 600 in FIG. 6).

In some embodiments, spatial coordinates of the examination position of the first target examination operation performed through the first extended reality device 120 (e.g., the second wearable sensory device or the digital twin) may be converted to spatial coordinates of the corresponding position on the patient. The coordinate conversion may be performed by the first extended reality device 120 or the physical examination device 110. A conversion relationship may be determined based on a coordinate system of the first extended reality device 120 and a coordinate system of the physical examination device 110. Moreover, the conversion relationship may be determined based on a ratio between a size of the digital twin and a size of the patient's body, or a ratio between a size of an object wearing the second wearable sensory device and a size of the patient's body.

The beneficial effects that may be brought by the embodiments of the present disclosure include but are not limited to the followings. (1) Using the patient's digital twin as a medium to realize remote physical examination, the operator can comprehensively and intuitively understand the patient's truc physical state through the digital twin. (2) Through the touch emulating device, the operator can experience a real touch of physical examination. Having thus described the basic concepts, it may be rather apparent to those skilled in the art after reading this detailed disclosure that the foregoing detailed disclosure is intended to be presented by way of example only and is not limiting. Various alterations, improvements, and modifications may occur and are intended to those skilled in the art, though not expressly stated herein. These alterations, improvements, and modifications are intended to be suggested by this disclosure, and are within the spirit and scope of the exemplary embodiments of this disclosure.

Moreover, certain terminology has been used to describe embodiments of the present disclosure. For example, the terms “one embodiment,” “an embodiment,” and/or “some embodiments” mean that a particular feature, structure or characteristic described in connection with the embodiment is included in at least one embodiment of the present disclosure. Therefore, it is emphasized and should be appreciated that two or more references to “an embodiment” or “one embodiment” or “an alternative embodiment” in various portions of this specification are not necessarily all referring to the same embodiment. Furthermore, the particular features, structures or characteristics may be combined as suitable in one or more embodiments of the present disclosure.

Further, it will be appreciated by one skilled in the art, aspects of the present disclosure may be illustrated and described herein in any of a number of patentable classes or context including any new and useful process, machine, manufacture, or composition of matter, or any new and useful improvement thereof. Accordingly, aspects of the present disclosure may be implemented entirely hardware, entirely software (including firmware, resident software, micro-code, etc.) or combining software and hardware implementation that may all generally be referred to herein as a “unit,” “module,” or “system.” Furthermore, aspects of the present disclosure may take the form of a computer program product embodied in one or more computer readable media having computer readable program code embodied thereon.

A computer readable signal medium may include a propagated data signal with computer readable program code embodied therein, for example, in baseband or as part of a carrier wave. Such a propagated signal may take any of a variety of forms, including electro-magnetic, optical, or the like, or any suitable combination thereof. A computer readable signal medium may be any computer readable medium that is not a computer readable storage medium and that may communicate, propagate, or transport a program for use by or in connection with an instruction execution system, apparatus, or device. Program code embodied on a computer readable signal medium may be transmitted using any appropriate medium, including wireless, wireline, optical fiber cable, RF, or the like, or any suitable combination of the foregoing.

Computer program code for carrying out operations for aspects of the present disclosure may be written in any combination of one or more programming languages, including an object oriented programming language such as Java, Scala, Smalltalk, Eiffel, JADE, Emerald, C++, C#, VB. NET, Python or the like, conventional procedural programming languages, such as the “C” programming language, Visual Basic, Fortran 2003, Perl, COBOL 2002, PHP, ABAP, dynamic programming languages such as Python, Ruby and Groovy, or other programming languages. The program code may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider) or in a cloud computing environment or offered as a service such as a Software as a Service (Saas).

Furthermore, the recited order of processing elements or sequences, or the use of numbers, letters, or other designations therefore, is not intended to limit the claimed processes and methods to any order except as may be specified in the claims. Although the above disclosure discusses through various examples what is currently considered to be a variety of useful embodiments of the disclosure, it is to be understood that such detail is solely for that purpose, and that the appended claims are not limited to the disclosed embodiments, but, on the contrary, are intended to cover modifications and equivalent arrangements that are within the spirit and scope of the disclosed embodiments. For example, although the implementation of various components described above may be embodied in a hardware device, it may also be implemented as a software only solution, e.g., an installation on an existing server or mobile device.

Similarly, it should be appreciated that in the foregoing description of embodiments of the present disclosure, various features are sometimes grouped together in a single embodiment, figure, or description thereof for the purpose of streamlining the disclosure aiding in the understanding of one or more of the various embodiments. This method of disclosure, however, is not to be interpreted as reflecting an intention that the claimed subject matter requires more features than are expressly recited in each claim. Rather, claimed subject matter may lie in less than all features of a single foregoing disclosed embodiment.

1. A method for remote physical examination, implemented on a physical examination device provided at a patient end, the method comprising:
obtaining physical information of a patient;
sending the physical information to a first extended reality device provided at an operator end, so that the first extended reality device generates a digital twin model of the patient based on the physical information;
receiving examination information from the first extended reality device, the examination information relating to a first target examination operation performed, based on the digital twin model through the first extended reality device, by an operator; and
performing a second target examination operation on the patient based on the examination information.

2. The method of claim 1, wherein the physical examination device includes a sensor configured to acquire the physical information.

3. The method of claim 1, wherein
the physical examination device includes a first wearable sensory device that is worn by the patient and configured to perform the physical examination on the patient; and/or
the physical examination device includes a mechanical arm configured to perform the physical examination on the patient.

4. (canceled)

5. The method of claim 1, wherein the examination information includes at least one examination position of the first target examination operation and at least one of a type, a direction, or a first intensity of the first target examination operation.

6. The method of claim 1, wherein the examination information includes at least one examination position of the first target examination operation and a first intensity of the first target examination operation; and
performing the second target examination operation on the patient based on the examination information includes:
determining whether the first intensity is within a protection range;
in response to determining that the first intensity is outside the protection range, determining a second intensity based on the protection range; and
performing the second target examination operation on the patient based on the second intensity.

7. The method of claim 6, wherein the protection range is determined using a protection range determination model based on at least one of patient information or medical records of the patient.

8. The method of claim 1, wherein
the examination information includes at least one examination position of the first target examination operation and at least one of a type of the first target examination operation, a direction of the first target examination operation, or a second intensity corresponding to the at least one examination position; and
the second intensity is determined, by the first extended reality device, based on a first intensity of the first target examination operation and a protection range.

9. The method of claim 1, wherein the digital twin model includes visualization of at least one diseased part of the patient, the at least one diseased part including at least one of a suspected diseased part or a definite diseased part.

10. The method of claim 9, wherein the suspected diseased part is predicted using a diseased part prediction model based on at least one of patient information or medical records of the patient.

11-13. (canceled)

14. A method for remote physical examination, implemented on a first extended reality device provided at an operator end, the method comprising:
obtaining physical information of a patient;
generating a digital twin model of the patient based on the physical information;
causing the digital twin model to be displayed;
determining examination information based on a first target examination operation performed, based on the digital twin model, by an operator; and
sending the examination information to a physical examination device provided at a patient end, so that the physical examination device performs a second target examination operation on the patient based on the examination information.

15. The method of claim 14, wherein the examination information includes at least one examination position of the first target examination operation and at least one of a type, a direction, or a first intensity of the first target examination operation.

16. The method of claim 14, wherein
the examination information includes at least one examination position of the first target examination operation and at least one of a type of the first target examination operation, a direction of the first target examination operation, or a second intensity corresponding to the at least one examination position; and
determining the examination information based on the first target examination operation includes:
determining whether a first intensity of the first target examination operation is within a protection range; and
in response to determining that the first intensity is outside the protection range, determining the second intensity based on the protection range.

17. The method of claim 16, wherein the protection range is determined using a protection range determination model based on at least one of patient information or medical records of the patient.

18. The method of claim 14, wherein
the first extended reality device includes a virtual touch emulating device worn by at least one hand of the operator; and
the method further comprises:
simulating, based on the examination information, a tactile sensation obtained by the operator when the first target examination operation or the second target examination operation is performed on the patient.

19. The method of claim 1, wherein the operator performs the first target examination operation on the digital twin model.

20. The method of claim 1, wherein the first extended reality device includes a second wearable sensory device.

21. The method of claim 20, wherein the operator performs the first target examination operation on the second wearable sensory device.

22. The method of claim 1, wherein the digital twin model includes visualization of at least one diseased part of the patient, the at least one diseased part including at least one of a suspected diseased part or a definite diseased part.

23. The method of claim 22, wherein the suspected diseased part is determined using a diseased part prediction model based on at least one of patient information or medical records of the patient.

24-26. (canceled)

27. A system for remote physical examination, comprising an operator end and a patient end, the operator end including a first extended reality device, the patient end including a physical examination device, wherein
the physical examination device is configured to
obtain physical information of a patient;
send the physical information to the first extended reality device;
receive examination information from the extended reality device; and
perform a second target examination operation on the patient based on the examination information; and
the first extended reality device is configured to receive the physical information from the physical examination device;
generate a digital twin model of the patient based on the physical information;
cause the digital twin model to be displayed;
determine the examination information based on a first target examination operation performed, based on the digital twin model through the first extended reality device, by an operator, and
send the examination information to the physical examination device.
